SEQUENCE LISTING (4 sequences)

SEQ ID NO: 1 - DNA, 24 bp, Artificial Sequence
  synthetic sequence
actgggattagataccccactatg

SEQ ID NO: 2 - DNA, 19 bp, Artificial Sequence
  synthetic sequence
accagctatcaccmrgctc

SEQ ID NO: 3 - DNA, 21 bp, Artificial Sequence
  synthetic sequence
cccactatgcytrgccctaaa

SEQ ID NO: 4 - protein, 23 aa, Artificial Sequence
  synthetic sequence
GTAYRCTTACCWTGTTACGACTT

(19) United States
(12) Patent Application Publication
(10) Pub. No.: US 2023/0085736 A1
(43) Pub. Date: Mar. 23, 2023

(54) PRIMER FOR DETECTING FECAL POLLUTION IN WATER, KIT AND HIGH-THROUGHPUT TRACING METHOD

(71) Applicant: NANJING UNIVERSITY, Nanjing (CN)

(72) Inventors: Xiwei HE, Nanjing (CN); Xuxiang ZHANG, Nanjing (CN); Jie GAO, Nanjing (CN); Zhaodong QI, Nanjing (CN)

(21) Appl. No.: 16/958,737

(22) PCT Filed: Nov. 15, 2019
(86) PCT No.: PCT/CN2019/118748

§ 371 Date: Jun. 29, 2020

(30) Foreign Application Priority Data
Sep. 19, 2019 (CN) 201910887484.9

(51) Int. Cl. C12Q 1/6888

(52) U.S. Cl. CPC C12Q 1/6888; C12Q 1/6869

(57) ABSTRACT

A primer for detecting fecal pollution in water, kit and high-throughput tracing method is disclosed, and belongs to the field of water pollution detection. The method mainly comprises the following steps: extracting DNA from water samples to be tested; using two pairs of universal primers for amplifying mitochondrial DNA to perform nested PCR amplification on the water sample DNA; performing high-throughput sequencing of the amplified products; performing annotation and alignment between sequencing data and a mitochondrial DNA database, and determining the sources of fecal pollution in water samples based on the alignment results. According to the present invention, nested PCR technology is utilized to amplify mitochondrial DNA with high sensitivity. The universal primers are combined with high-throughput sequencing, which can not only trace multiple sources of potential fecal pollution simultaneously, but also correspondingly quantify the degree of fecal pollution, thereby determining the main pollution source.

### CROSS REFERENCE TO RELATED APPLICATION

This application is a national stage application of International application number PCT/CN2019/118748, filed Nov. 15, 2019, titled “PRIMER FOR DETECTING FECAL POLLUTION IN WATER, KIT AND HIGH-THROUGHPUT TRACING METHOD”, which claims the priority benefit of Chinese Patent Application No. 201910887484.9, filed Sep. 19, 2019, which is hereby incorporated by reference in its entirety.

### TECHNICAL FIELD

The present invention relates to a method for rapidly tracing the sources of fecal pollution in water, and more particularly, to a method for simultaneously screening various potential fecal pollution by tracking mitochondrial DNA carried in animal feces as a marker, using nested PCR combined with high-throughput sequencing technology.

### BACKGROUND

In recent years, with the continuous growth of population and economy, water pollution is getting worse, especially fecal pollution. When fecal pollutants enter into the water, they not only increase the content of nitrogen and phosphorus in the water, causing eutrophication, but also bring pathogenic bacteria that may exist in feces into the water, causing the spread of diseases. Therefore, it is urgent to prevent and control fecal pollution in water.

There are many potential sources of fecal pollution, including domestic sewage, livestock wastewater, pet and wildlife excreta, etc. Due to ineffective treatment, these pollutants are often directly discharged into the environment, resulting in fecal pollution of the water. In the case of unknown pollution incidents, lack of accurate identification of the fecal sources often makes the pollution abatement inefficient and costly. Therefore, accurate identification of the sources of fecal pollution in water has become the key to solve the problem.

At present, the source marker method has been widely used to trace the sources of fecal pollution. That is, fecal pollution in water is identified by detecting markers indicative of the fecal sources. The most studied markers include chemical markers and microbial markers, but some of the existing chemical and microbial markers have low specificity, which often leads to false positive results. In recent years, researchers have found that there are a large number of mitochondrial DNA from exfoliated intestinal cells in feces. The rich mononucleotide polymorphic sites in specific fragments of mitochondrial DNA makes it species specific, which can be used for the purpose of tracing the sources of fecal pollution by detecting species-specific mitochondrial DNA fragments in the fecal polluted water. Meanwhile, there are some conserved regions in mitochondrial DNA fragments, making it possible to design universal polymerase chain reaction (PCR) primers to simultaneously amplify mitochondrial DNA fragments of different species.

Through retrieval, we found that relevant applications have been disclosed in the prior art. For example, Chinese patent application No. 201510300317.1, published on Sep. 2, 2015, discloses a nested PCR primer, kit and method for detecting duck feces pollution in water by using the kit containing the nested PCR primer. The method comprises the following steps: (1) extracting DNA from water sample to be tested; (2) performing the first round of PCR amplification by taking the water sample DNA as a template, wherein PCR primers are SDF and SDR; (3) after the first round of PCR amplification, performing the second round of PCR amplification and taking PCR products as templates, wherein PCR primers are NDF and NDR; and (4) performing gel electrophoresis, observing whether there is a band of 158 bp in the products obtained in the second round of PCR, if it exsits, it indicates that the water is contaminated by duck feces. Although the above method can accurately screen whether duck feces pollution exists, the application range of the method is narrow, which limits its application in more complicated feces pollutions.

Also, Chinese patent application No. 201510305364.5, published on Aug. 19, 2015, discloses a PCR kit for simultaneously detecting dog, pig and chicken feces pollution in water and a detection method thereof. The kit contains specific primers for the above pollution sources: primers 1 to 6. The mixed primers (6 kinds of primers) are used for amplification reaction in the same PCR system, making it possible to distinguish among three fecal pollution sources of pigs, dogs and chickens in the PCR reaction system. The detection method is as follows: extracting DNA from water sample to be tested, using it as a template, and performing PCR amplification by using primers, dNTP, PCR buffer, Taq enzyme, Mg′ solution and ddH₂O provided in a kit; and observing PCR products under the ultraviolet lamp after agarose gel electrophoresis. If there are bands of 390 bp, 490 bp and 783 bp, they respectively indicate the dog, pig and chicken feces pollution in water. The kit can accurately detect the dog, pig and chicken feces pollution in water alone or in random combination. Although the method of the above application can detect three kinds of fecal pollution simultaneously, the detection range is still relatively limited, and high-throughput detection is not possible for water pollution with numerous corresponding pollution sources. In addition, it is impossible to relatively quantify the degree of fecal pollutions of different sources, thus limiting the application for fecal source tracking in water.

In view of the problems that there are many potential sources of fecal pollution in water, it is urgent to provide a high-throughput method for quick and accurate tracing of as many as possible potential sources of fecal pollution.

### SUMMARY

### 1. Problems to be Solved

In view of the problems that there are many potential sources of fecal pollution in water and it is difficult to identify the pollution sources, the present invention provides universal nested PCR primers capable of amplifying multiple species-specific mitochondrial DNA fragments, and a method for high-throughput tracing of the fecal sources (human, livestock, wild birds, mammals and the like) by using nested PCR combined with high-throughput sequencing technology, which can achieve the purpose of quick and accurate identification of the sources of fecal pollution in water.

### 2. Technical Solution

In order to solve the foregoing problems, the technical solutions adopted by the present invention are as follows:

The present invention provides a primer for detecting fecal pollution in water, wherein the primer has nested PCR amplification sequences, and the sequences are respectively:

AF (5′-3′):
(SEQ ID NO: 1)
ACTGGGATTAGATACCCCACTATG;
AR (5′-3′):
(SEQ ID NO: 2)
ACCAGCTATCACCMRGCTC;
BF (5′-3′):
(SEQ ID NO: 3)
CCCACTATGCYTRGCCCTAAA;
and
BR (5′-3′):
(SEQ ID NO: 4)
GTAYRCTTACCWTGTTACGACTT.

The present invention further provides a kit for detecting fecal pollution in water, comprising the primer for detecting fecal pollution in water, dNTP, PCR buffer, Taq enzyme, Mg²⁺ solution and ddH₂O.

As a further improvement of the present invention, the PCR buffer is 10×PCR buffer, and the concentration of the dNTP is 2.5 mM, the concentration of the Taq enzyme is 5 U/μL, and the concentration of the Mg²⁺ solution is 25 mmol/L.

As a further improvement of the present invention, the present invention further provides a high-throughput method for detecting the sources of fecal pollution in water, comprising the following steps:

(1) taking water samples to be tested, filtering the water samples through a mixed cellulose ester membrane with the pore diameter of 0.22 μm and then collecting the filter membrane, and extracting DNA from the surface retentate; and

(2) performing nested PCR amplification on the water sample DNA by using primers AF and AR, BF and BR, respectively; wherein the sequences of the two pairs of primers are as follows:

AF (5′-3′):
(SEQ ID NO: 1)
ACTGGGATTAGATACCCCACTATG;
AR (5′-3′):
(SEQ ID NO: 2)
ACCAGCTATCACCMRGCTC;
BF (5′-3′):
(SEQ ID NO: 3)
CCCACTATGCYTRGCCCTAAA;
and
BR (5′-3′):
(SEQ ID NO: 4)
GTAYRCTTACCWTGTTACGACTT;

first of all, using AF and AR for the first round of PCR amplification, and taking the water sample DNA as a template, wherein the amplification conditions are: predenaturation for 5 min at 95° C., for 30 s at 95° C., for 60 s at 59° C., for 45 s at 72° C., 35 cycles; for 7 min at 72° C., preservation at 4° C.; then using BF and BR for the second round of PCR amplification, and taking the products in the first round of PCR as templates, wherein the amplification conditions are: predenaturation for 5 min at 95° C., for 30 s at 95° C., for 40 s at 57° C., for 45 s at 72° C., 35 cycles; for 7 min at 72° C., preservation at 4° C.;

(3) recovering and purifying the nested PCR products, followed by performing high-throughput sequencing, wherein the sequencing data is returned in .fastq format; removing the short-length sequences and converting the data into .fasta format;

(4) constructing the mitochondrial DNA database of various species, performing BLAST alignment between the sequences obtained in step (3) and the mitochondrial DNA database, and deeming the species returned by BLAST as potential fecal pollution sources; wherein when the number of the returned species is equal to or greater than 2, the proportion of the number of annotated sequences of each species to the total sequence number is positively correlated with the degree of fecal pollution from each source.

As a further improvement of the present invention, in step (3), the 500 bp-length PCR product is recovered for purification, and the sequences with a length less than 450 bp are removed after high-throughput sequencing.

As a further improvement of the present invention, in step (4), the mitochondrial DNA database is a mitochondrial 12S rRNA gene library, and the BLAST annotation threshold is 0.8.

### 3. Beneficial Effects

Compared with the prior art, the present invention has the following beneficial effects: (1) The primer for detecting fecal pollution in water according to the present invention can simultaneously amplify mitochondrial genes of various species, including human beings, livestock, wild birds and mammals. The primer has broad spectrum; at the same time, the DNA sequence amplified by the primer contains species-specific fragments of various species. The species can be identified through sequence alignment, and a high-throughput tracing of the sources of fecal pollution (such as Homo sapiens, Sus scrofa, Bos taurus, Ovis aries, Gallus gallus, Anas platyrhynchos, Anser cygnoides, Felis catus, Mus musculus and Gracula religiosa) can be realized with the high-throughput sequencing technology.

(2) The primer for detecting fecal pollution in water according to the present invention is applicable to different species, which is used for nested PCR amplification to ensure that the lengths of products obtained in the first and the second round of PCR are moderate, and combined with high-throughput sequencing technology, which can be used to perform qualitative screening on a plurality of potential fecal pollution sources simultaneously; for the water with complicated fecal pollution conditions, the sources of fecal pollution can be identified soon, thereby saving time and costs.

(3) The high-throughput tracing method for detecting fecal pollution in water according to the present invention takes mitochondrial DNA as a marker of fecal pollution sources, which has higher source specificity than conventional chemical and microbial markers, to effectively reduce false positive results. This method also has a high sensitivity for tracing the sources of fecal pollution in water, and can detect micro fecal pollution in water.

(4) In the high-throughput tracing method for detecting fecal pollution in water according to the present invention, universal primers that can simultaneously amplify mitochondrial genes of various species are designed and the mitochondrial DNA database is constructed for alignment and identification; in combination with high-throughput sequencing, the method can not only be used for qualitatively tracing the sources of various potential fecal pollution, but also be used for relative quantitative screening of the degrees of various fecal pollution, thus determining main pollution sources.

### BRIEF DESCRIPTION OF DRAWINGS

FIG. 1 is a schematic diagram of the high-throughput method for tracing the sources of fecal pollution in water according to the present invention;

FIG. 2 is a PCR amplification diagram of the universal primers according to the present invention on DNA of water samples contaminated by feces from different sources, wherein (A) is a PCR amplification diagram of universal primers AF and AR; and (B) is a PCR amplification diagram of universal primers BF and BR; and

FIG. 3 is a sequence information diagram showing high-throughput sequencing data.

### DETAILED DESCRIPTION

Unless otherwise specified, the terms used in the present invention have the meanings commonly understood by those of ordinary skill in the art. The present invention is further described in detail below with reference to specific examples and the accompanying drawings. It should be noted that these examples are only for the purpose of illustrating the present invention and are not intended to limit the scope of the present invention in any way.

### Example 1

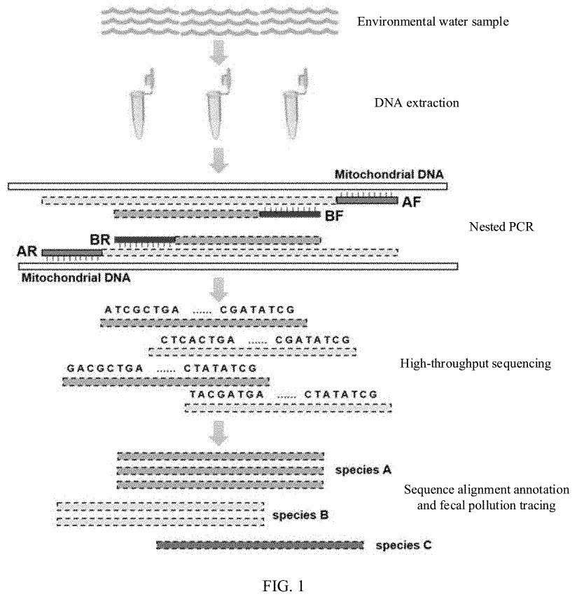

A method of the present invention can simultaneously detect fecal pollution from sources including human beings, livestock, wild birds, mammals and the like, and the steps are shown in FIG. 1. In this example, Homo sapiens, Sus scrofa, Bos taurus, Ovis aries, Gallus gallus, Anas platyrhynchos, Anser cygnoides, Felis catus, Mus musculus and Gracula religiosa were selected as representatives, and fecal pollution water samples of various species were simulated in the laboratory to verify the applicability of universal primers in the present invention.

Fresh feces of the above species were collected, a small amount of the feces were respectively added into river water which was not contaminated by feces, and 1 L of the feces-containing river water was respectively taken and filtered through a mixed cellulose ester membrane with a pore diameter of 0.22 μm; and then DNA on the filter membrane was extracted by using FastDNA SPIN Kit for Soil (Takara), and the DNA was taken as the template DNA to perform nested PCR amplification. The PCR instrument was ABI Veriti96 instrument. FIG. 2 is a PCR amplification diagram of universal primers according to the present invention on DNA of water samples contaminated by feces from different sources. First, two rounds of PCR amplification were performed.

The primer sequences of the first round of PCR were: AF (5′-3′): ACTGGGATTAGATACCCCACTATG (SEQ ID NO: 1) and AR (5′-3′): ACCAGCTATCACCMRGCTC (SEQ ID NO: 2); PCR reaction system was: 2.5 μl of 10×PCR buffer; 2.5 μL of dNTP; 0.1 μL of Taq enzyme; 2 μL of Mg²⁺; 0.5 μL of AF primer, 0.5 μL of AR primer, 2 μL of template DNA, making up to 25 μL with sterile water; and PCR reaction conditions were: predenaturation for 5 min at 95° C., for 30 s at 95° C., for 60 s at 59° C., for 45 s at 72° C., 35 cycles; for 7 min at 72° C., preservation at 4° C.

The products in the first round of PCR were subjected to 1% gel electrophoresis at 120 V and 440 mA for 25 min. The electrophoresis results are shown in FIG. 2A. DNA of water samples contaminated by feces from various species could be amplified by universal primers AF and AR, and the amplified fragments had the same length (about 950 bp).

Then, the products in the first round of PCR were used as templates to perform the second round of PCR amplification, and the PCR primers were BF (5′-3′): CCCACTATGCYTRGCCCTAAA (SEQ ID NO: 3) and BR (5′-3′): GTAYRCTTACCWTGTTACGACTT (SEQ ID NO: 4); PCR reaction system was: 2.5 μL of 10×PCR buffer; 2.5 μL of dNTP; 0.1 μL of Taq enzyme; 2 μL of Mg²⁺; 0.5 μL of BF primer, 0.5 μL of BR primer, 2 μL of template DNA, making up to 25 μL with sterile water; and PCR reaction conditions were: predenaturation for 5 min at 95° C., for 30 s at 95° C., for 40 s at 58° C., for 45 s at 72° C., 35 cycles; for 7 min at 72° C., preservation at 4° C.

The products in the second round of PCR were subjected to 1% gel electrophoresis at 120 V and 440 mA for 25 min. The electrophoresis results are shown in FIG. 2B. It can be seen that the universal nested PCR primers in the present invention could be used to amplify the DNA of water samples contaminated by feces from various species, and the nested PCR products of each species had the same length (about 500 bp).

The above results show that the nested PCR primers in the present invention has good applicability to different species, and the length of the nested PCR products are moderate, which is favorable for performing high-throughput sequencing by using the current mainstream second generation sequencing platform.

### Example 2

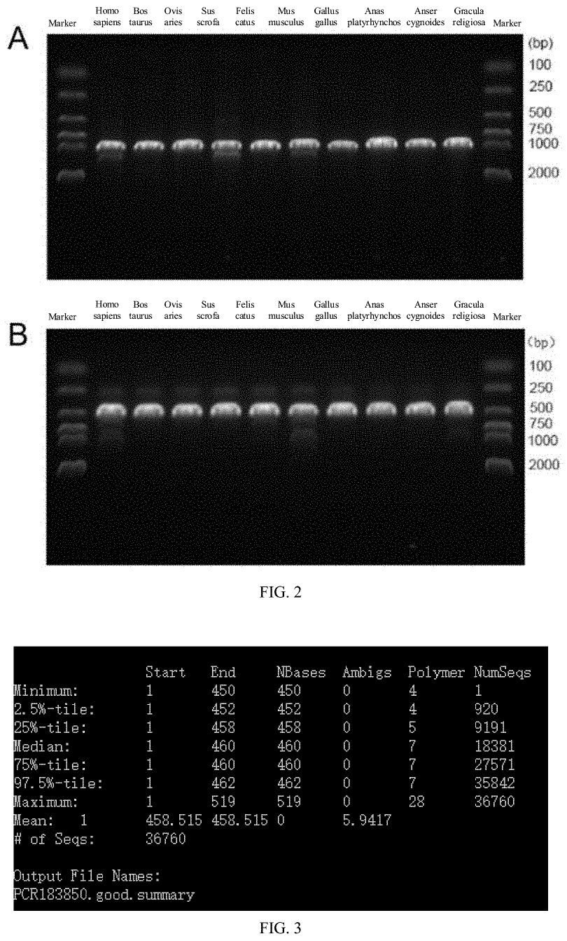

The feces of the above 10 species were used to simulate the detection of water contaminated by multiple feces. Ten kinds of feces were simultaneously added into river water which was not contaminated by feces, wherein the Bos taurus feces were added in the largest amount. One liter of the above river water was taken and filtered by a mixed cellulose ester membrane with a pore diameter of 0.22 μm; then DNA on the filter membrane was extracted by using FastDNA SPIN Kit for Soil (MP bio), and the DNA was taken as the template DNA to perform nested PCR amplification in the same way as in Example 1. The nested PCR products were subjected to gel electrophoresis in the same way as in Example 1. The PCR product at 500 bp was recovered from gel and purified by Takara MiniBEST Agarose Gel DNA Extraction Kit (Takara), and high-throughput sequencing was performed by an IlluminaMiseq sequencing platform, with the read length mode of 300 bp at both ends. The obtained sequencing data in .fastq format was first subjected to quality control by prinseq-lite software to ensure that the quality value of each base was greater than 30. The data format was converted to .fasta, and then sequences with the length less than 450 bp were removed by mothur software to obtain 36760 valid sequences. The sequence information is shown in FIG. 3.

In order to verify the accuracy of sequencing results, a simple mitochondrial DNA database was constructed, which contained mitochondrial 12S rRNA genes of 20 species (Table 1). The annotation and alignment between the sequencing data and the simple database was performed by local BLAST, and the BLAST annotation threshold was set to 0.8.

The species information of the simple mitochondrial DNA database and the results of numbers of annotated sequences are shown in Table 1, with a total of 36160 sequences successfully annotated. The annotated species were Homo sapiens, Sus scrofa, Bos taurus, Ovis aries, Gallus gallus, Anas platyrhynchos, Anser cygnoides, Felis catus, Mus musculus and Gracula religiosa, which were completely consistent with the species sources of feces contained in the water sample, while none of the other species was annotated, indicating that the method of the present invention has good accuracy and specificity. In addition, among all annotated sequences, the number of sequences derived from Bos taurus was the largest, indicating that Bos taurus feces pollution was relatively the most serious, which was consistent with the actual situation. It is indicated that the method of the present invention can be used to identify the main pollution source in a complex fecal pollution environment.

The species returned by BLAST were taken as potential fecal pollution sources. When the number of returned species was equal to or greater than 2, the proportion of the number of annotated sequences of each species to the total sequence number is positively correlated with the degree of fecel pollution from each source.

TABLE 1
Species information of the simple mitochondrial
DNA database and numbers of annotated sequences

| | NCBI serial | Number of annotated |
|---|---|---|
| Species | number | sequences |
| Cygnus | NC_027095.1 | 0 |
| Phoca largha | FJ895151.1 | 0 |
| Castor canadensis | NC_033912.1 | 0 |
| Anser cygnoides | KU211647.1 | 53 |
| Meles leucurus amurensis | KU707936.1 | 0 |
| Egretta eulophotes | EU072995.1 | 0 |
| Gracula religiosa | JF937590.1 | 5852 |
| Phalacrocorax carbo | NC_027267.1 | 0 |
| Anas platyrhynchos | MF069251.1 | 3033 |
| Equus caballus | KX669268.1 | 0 |
| Felis catus | NC_001700.1 | 1209 |
| Bos taurus | MK028750.1 | 19976 |
| Canis lupus familiaris | HM048871.1 | 0 |
| Homo sapiens | FJ986465.1 | 2799 |
| Sciurus vulgaris | KU962990.1 | 0 |
| Mus musculus | EU450583.1 | 36 |
| Ursus arctos | AF303110.1 | 0 |
| Ovis aries | KU575248.1 | 3117 |
| Mustek itatsi | NC_034330.1 | 0 |
| Gallus gallus | KX512321.1 | 52 |
| Sus scrofa | KX146493.1 | 33 |
| Total | | 36160 |

The present invention and its embodiments have been schematically described above, and the description is not restrictive. The accompanying drawing also shows only one embodiment of the present invention, and the actual structure is not limited thereto. Therefore, if a person of ordinary skill in the art designs similar structural modes and embodiments without creativity under the enlightenment without departing from the purpose of the present invention, the structural modes and the embodiments should fall within the protection scope of the present invention.

### Sequence Listing

SEQ ID NO: 1
ACTGGGATTAGATACCCCACTATG
SEQ ID NO: 2
ACCAGCTATCACCMRGCTC
SEQ ID NO: 3
CCCACTATGCYTRGCCCTAAA
SEQ ID NO: 4
GTAYRCTTACCWTGTTACGACTT

What is claimed is:

1. A primer for detecting fecal pollution in water, wherein the primer has nested PCR amplification sequences, and the sequences are respectively:
AF (5′-3′):
(SEQ ID NO: 1)
ACTGGGATTAGATACCCCACTATG;
AR (5′-3′):
(SEQ ID NO: 2)
ACCAGCTATCACCMRGCTC;
BF (5′-3′):
(SEQ ID NO: 3)
CCCACTATGCYTRGCCCTAAA;
and
BR (5′-3′):
(SEQ ID NO: 4)
GTAYRCTTACCWTGTTACGACTT.

2. A kit for detecting fecal pollution in water, comprising the primer for detecting fecal pollution in water according to claim 1, dNTP, PCR buffer, Taq enzyme, Mg²⁺ solution and ddH₂O.

3. The kit for detecting fecal pollution in water according to claim 2, wherein the PCR buffer is 10×PCR buffer, and the concentration of the dNTP is 2.5 mM, the concentration of the Taq enzyme is 5 U/μL, and the concentration of the Mg²⁺ solution is 25 mmol/L.

4. A high-throughput traceable method for detecting fecal pollution in water, comprising the following steps:
(a) sample treatment: taking water samples to be tested and extracting DNA from the water samples;
(b) nested PCR amplification: performing nested PCR amplification on the DNA extracted in step (a) to obtain nested PCR products, by using the primer for detecting fecal pollution in water according to claim 1 or the kit for detecting fecal pollution in water according to any one of claims 2-3;
(c) high-throughput sequencing: recovering and purifying the nested PCR products, and then performing high-throughput sequencing, outputting the sequencing results in .fastq format, and filtering out the sequences with short sequencing length; and
(d) pollution source analysis: performing BLAST alignment between that sequences obtained in step (c) and a mitochondrial DNA database, and analyzing pollution sources according to BLAST annotation information.

5. The high-throughput traceable method for detecting fecal pollution in water according to claim 4, wherein the process of the nested PCR in step (b) comprises the following steps: first performing the first round of PCR amplification by AF and AR, wherein the water sample DNA is used as the template, and the amplification conditions are as follows: predenaturation for 5 min at 95° C., for 30 s at 95° C., for 60 s at 59° C., for 45 s at 72° C., 35 cycles; for 7 min at 72° C., preservation at 4° C.; and then performing the second round of PCR amplification by BF and BR, wherein the products in the first round of PCR are used as the templates, and the amplification conditions are as follows: predenaturation for 5 min at 95° C., for 30 s at 95° C., for 40 s at 58° C., for 45 s at 72° C., 35 cycles; for 7 min at 72° C., preservation at 4° C.

6. The high-throughput traceable method for detecting fecal pollution in water according to claim 5, wherein in step (c), the product at 500 bp is recovered for purification, and the sequences with a length less than 450 bp are filtered out after high-throughput sequencing.

7. The high-throughput traceable method for detecting fecal pollution in water according to claim 6, wherein in step (d), the mitochondrial DNA database is a mitochondrial 12S rRNA gene library.

8. The high-throughput traceable method for detecting fecal pollution in water according to claim 7, wherein the BLAST annotation threshold is 0.8.

9. The high-throughput traceable method for detecting fecal pollution in water according to claim 8, wherein in step (d), the species returned by BLAST are taken as potential fecal pollution sources, and when the number of the returned species is equal to or greater than 2, the proportion of the number of annotated sequences of each species to the total sequence number is positively correlated with the degree of fecal pollution from each source.
